(19) United States
(12) Patent Application Publication
(10) Pub. No.: US 2007/0117851 A1
(43) Pub. Date: May 24, 2007

(54) Liquid pharmaceutical compositions of nimodipine

(75) Inventors: Julius Remenar, Framingham, MA (US); Zhong Zhang, Sudbury, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: TRANSFORM PHARMACEUTICALS, INC., Lexington, MA (US)

(21) Appl. No.: 11/548,880

(22) Filed: Oct. 12, 2006

Related U.S. Application Data
(63) Continuation of application No. 60/727,212, filed on Oct. 14, 2005.

(51) Int. Cl. A61K 31/455

(52) U.S. Cl. 514/355

(57) ABSTRACT

The present invention relates to liquid compositions of nimodipine and administration of said compositions. New stable liquid compositions of nimodipine can be used to treat conditions such as, but not limited to, aneurysms, subarachnoid hemorrhage, vasospastic angina, Prenzmetal's angina, stable angina, acute myocardial infarction, myocardial arrest, arrhythmia, systemic hypertension, pulmonary hypertension, congestive heart failure, and hypertrophic cardiomyopathy.

### BACKGROUND OF THE INVENTION

Nimodipine is a dihydropyridine derivative with the name 1,4-dihydro-2,6-dimethyl-4-(3-nitrophenyl)-3,5-pyridinedicarboxylic acid 2-methoxyethyl 1-methylethyl ester. Nimodipine acts as a vasodilator and is considered to be a calcium channel blocker. Nimodipine has poor aqueous solubility. The structure of nimodipine is shown below as Formula I:

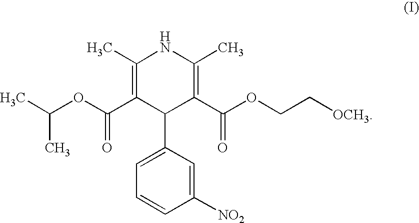

Conventionally, nimodipine is orally administered via a swallowable dosage form, such as NIMOTOP® liquid-filled capsules (Bayer Pharmaceuticals Corp.). NIMOTOP® capsules each contain 30 mg of nimodipine and are commonly administered in a two-capsule 60 mg dose. However, there also exist occasions when medical professionals need to administer nimodipine to a patient who finds it difficult or is unable to swallow capsules, such as when a patient is unconscious. Under such circumstances, nimodipine can be administered via an intraoral or an intranasal (e.g., naso-gastric) tube. Currently, there exists an unmet need in the field for an easily-administrable liquid nimodipine dosage forms for patients who find it difficult or are unable to swallow.

Currently administration of nimodipine via an intraoral or an intranasal tube requires medical practitioners to withdraw the liquid nimodipine composition from one or two or more commercially available capsules via syringe or another device prior to administration. In such a circumstance, the practitioner may, either unknowingly or due to handling, extract less than the full amount of the liquid dose from the capsule. This procedure can introduce substantial risk of incomplete dosing while placing a greater burden on medical professionals. Incomplete dosing can be exacerbated by the relatively small dosage volumes and high drug concentration of the commercially available capsules. A practitioner's failure to dose the full amount of the high-concentration, small volume liquid from the commercial capsules could lead to a significant underdose of nimodipine. Additionally, the liquid to be extracted from commercially available capsules may have a viscosity that is too high to enable simple and accurate administration via an intraoral or an intranasal (e.g., naso-gastric) tube, due to the small diameter of such a tube.

As such, there exists a need for a readily-administrable, accurate liquid nimodipine composition with an optimal nimodipine concentration for patients who find it difficult or are unable to swallow.

While other liquid nimodipine pharmaceutical compositions exist in the art, such compositions, like the capsules discussed above, are not suitable. For example, U.S. Pat. No. 4,537,898 describes drop formulations of compounds including nimodipine comprising a high concentration (approximately 50 percent by weight or more) of ethanol. Such drop formulations could lead to similar dosing and administration difficulty as those described for the capsules. In addition, the high concentration of ethanol in the drops could lead to several problematic scenarios, such as inaccurate dosing caused by ethanol evaporation, extraction of leachable compounds from packaging, or flammability and evaporation issues during and after the manufacturing process. It would also be desirable to obtain an acceptable liquid nimodipine dosage form with a minimal amount of ethanol to limit the amount of ethanol being administered to an already compromised patient. The present invention provides liquid nimodipine in optimal concentration and optimal volume compositions for easy administration to patients via an intraoral or an intranasal tube with a relatively low ethanol concentration and/or a low amount of ethanol per dose.

### SUMMARY OF THE INVENTION

The present invention relates to novel liquid pharmaceutical compositions of nimodipine with improved properties such as higher concentrations of nimodipine with lower concentrations of ethanol than that in the art which makes it more suitable for administration to patients via an intraoral or intranasal tube.

In one aspect, the present invention provides a liquid composition comprising nimodipine, an alcohol, and a solvent.

In another aspect, the present invention provides a composition of nimodipine comprising a concentration great enough to administer at least about 60 mg in a single unit less than or equal to about 15 mL in volume.

In another aspect, the present invention provides a method of treating conditions such as, but not limited to, aneurysms, subarachnoid hemorrhage, vasospastic angina, Prenzmetal's angina, stable angina, acute myocardial infarction, myocardial arrest, arrhythmia, systemic hypertension, pulmonary hypertension, congestive heart failure, and hypertrophic cardiomyopathy with an improved liquid pharmaceutical composition of nimodipine.

In a first embodiment, the present invention provides a composition of nimodipine, wherein the concentration of nimodipine is at least about 5.00 mg/mL. For example, at least about 6.00 mg/mL nimodipine, at least about 7.00 mg/mL nimodipine, at least about 8.00 mg/mL nimodipine, at least about 9.00 mg/mL nimodipine, at least about 10.00 mg/mL nimodipine, at least about 11.00 mg/mL nimodipine, or at least about 12.00 mg/mL nimodipine.

In another embodiment, a composition of nimodipine comprises nimodipine, an alcohol, and a solvent, wherein the concentration of nimodipine is at least about 5.0 mg/mL. In a specific embodiment, the alcohol is ethanol. In another specific embodiment, the solvent is a non-ionic surfactant such as Cremophor® brand manufactured by BASF. In another specific embodiment, the solvent is a mixture of a non-ionic surfactant and propylene glycol. In another specific embodiment, the solvent is a mixture of a non-ionic surfactant and a polyethylene glycol. In another specific embodiment, the solvent is a mixture of a non-ionic surfactant, a polyethylene glycol, and propylene glycol.

In another embodiment, the present invention provides a composition of nimodipine, wherein said composition is prepared in a readily-dispensable container. Said readily-dispensable container can be used for administration of the composition, for example, via an intraoral or an intranasal tube.

In another embodiment, the present invention provides a 60 mg dose of nimodipine with about a 5-15 mL composition volume packaged in a single-use container. In another embodiment, said 60 mg dose is completely solubilized in a liquid composition. In another embodiment, said single-use container comprises about 60 mg nimodipine solubilized in a total composition volume which is less than or equal to about 15.00 mL, such as, for example, less than or equal to about 12.00 mL, less than or equal to about 10.00 mL, less than or equal to about 9.00 mL, less than or equal to about 8.00 mL, less than or equal to about 7.00 mL, less than or equal to about 6.00 mL, or less than or equal to about 5.00 mL.

In another embodiment, the present invention provides a pharmaceutical composition comprising nimodipine, an alcohol, and a solvent. In another embodiment, the present invention provides a medicament comprising nimodipine, an alcohol, and a solvent.

### BRIEF DESCRIPTION OF THE DRAWINGS

FIG. 1 shows the chemical stability of nimodipine in the composition described in Table 2 at 40 degrees C.

FIG. 2 shows the chemical stability of nimodipine in the composition described in Table 2 at 60 degrees C.

FIG. 3 shows several compositions of Cremophor® EL, ethanol, and ethanol/Cremophor® EL with varying amounts of excipient.

FIG. 4 shows several compositions according to the invention comprising ethanol, Cremophor® EL, and propylene glycol with varying amounts of propylene glycol.

FIG. 5 shows several compositions according to the invention comprising ethanol, Cremophor® EL, and PEG 400 with varying amounts of PEG 400.

FIG. 6 shows several compositions according to the invention comprising ethanol, Cremophor® EL, and 1:1 propylene glycol:PEG 400 with varying amounts of 1:1 propylene glycol:PEG 400.

FIG. 7 shows several compositions according to the invention comprising PEG 400, propylene glycol, 1:1 propylene glycol:PEG 400, and ethanol in varying amounts with 10% ethanol and 30% Cremophor® EL.

### DETAILED DESCRIPTION OF THE INVENTION

The present invention relates to improved liquid compositions of nimodipine. Such compositions of nimodipine comprise a concentration great enough to administer at least about 60 mg in a single unit less than or equal to about 15 mL in volume while minimizing the concentration and/or amount of ethanol in the composition. Nimodipine compositions may, optionally, be administered using a readily-dispensable container according to the present invention.

The present invention provides pharmaceutical compositions of nimodipine which are stable and can be used intraorally or intranasally to treat conditions such as, but not limited to, aneurysms, subarachnoid hemorrhage, vasospastic angina, Prenzmetal's angina, stable angina, acute myocardial infarction, myocardial arrest, arrhythmia, systemic hypertension, pulmonary hypertension, congestive heart failure, and hypertrophic cardiomyopathy.

Pharmaceutical compositions and medicaments may be described as mixtures of two or more components “by volume,” which is herein defined as the volume due to one component divided by the volume of all components of the pharmaceutical composition. This ratio may be converted to or reported as a percentage of the total composition volume. Such a quantity may also be indicated by “v/v” or “percent v/v.” Similarly, the phrase “by weight” describes the weight due to one component divided by the weight of all components of the composition. This ratio may be converted to or reported as a percentage of the total composition weight. Such a quantity may also be indicated by “w/w” or “percent w/w.”

As used herein, the term “nimodipine” includes the racemate, other mixtures of (+)- and (−)-isomers, and single enantiomers, but may be specifically set forth as the racemate, (+)-isomer, (−)-isomer, or any mixture of both (+)- and (−)-isomers.

In a first embodiment, liquid pharmaceutical compositions of the invention comprise nimodipine, an alcohol, and a solvent. These liquid compositions exist as a homogeneous aqueous phase.

In another embodiment, the present invention provides a liquid pharmaceutical composition comprising:

(a) nimodipine;

(b) an alcohol; and

(c) a solvent;

with the proviso that if said alcohol comprises ethanol, the ethanol concentration of said pharmaceutical composition is less than about 15 percent by weight.

In another embodiment, said liquid pharmaceutical composition further comprises water.

In certain embodiments, said pharmaceutical composition comprises about 60 mg nimodipine.

In a specific embodiment, said alcohol is ethanol. In another specific embodiment, said alcohol is propylene glycol. In certain embodiments, said alcohol comprises a mixture of two or more pharmaceutically acceptable alcohols. In a specific embodiment, said alcohol comprises a mixture of ethanol and propylene glycol. Further examples of alcohol include, but are not limited to, glycerol, 2-(2-ethoxyethoxy)-ethanol (e.g., TRANSCUTOL®, Gattefosse, Westwood, N.J. 07675), benzyl alcohol, and other pharmaceutically acceptable alcohols. Liquid pharmaceutical compositions of the present invention can also include mixtures of two or more of the aforementioned alcohols.

In certain embodiments, said solvent comprises a non-ionic surfactant, such as Cremophor® brand non-ionic surfactants. In certain embodiments, said solvent comprises a mixture of two or more solvents. In a specific embodiment, said solvent comprises non-ionic surfactant and polyethylene glycol, such as Cremophor® brand non-ionic surfactants and PEG 400. Polyethylene glycol is considered a solvent and not an alcohol according to the present invention. A solvent is a chemical substance capable of solubilizing nimodipine according to the present invention. Solvents include, but are not limited to, non-ionic surfactants and ionic surfactants. Several non-limiting examples of solvents include Cremophor® EL, Cremophor® RH, Vitamin E TPGS, polyethylene glycol, and Solutol® brand, such as Solutol® HS 15. Liquid pharmaceutical compositions of the present invention can also include mixtures of two or more of the aforementioned solvents. An alcohol, as described herein, is not considered to be a solvent according to the present invention. Water, although part of the composition, is not considered to be a solvent according to the present invention.

In another embodiment, the present invention provides a composition of nimodipine, wherein the nimodipine is present in an amount of at least about 60.00 mg in a single oral liquid dosage unit. In one embodiment, the concentration of nimodipine in the composition is sufficient to administer a 60.00 mg dose with a total composition volume of less than about 50 mL. In another embodiment, the concentration of nimodipine in the composition is sufficient to administer a 60.00 mg dose with a total composition volume of less than about 25 mL. In another embodiment, the concentration of nimodipine in the composition is sufficient to administer a 60.00 mg dose with a total composition volume of less than about 20 mL. In another embodiment, the concentration of nimodipine in the composition is sufficient to administer a 60.00 mg dose with a total composition volume of less than about 15 mL. In another embodiment, the concentration of nimodipine in the composition is sufficient to administer a 60.00 mg dose with a total composition volume of less than about 10 mL. In another embodiment, the concentration of nimodipine in the composition is sufficient to administer a 60.00 mg dose with a total composition volume of less than about 7 mL. For example, the total composition volume can be about 3.00 mL, 4.00 mL, 5.00 mL, 6.00 mL, 7.00 mL, 8.00 mL, 9.00 mL, 10.00 mL, 11.00 mL, 12.00 mL, 13.00 mL, 14.00 mL, or 15.00 mL, or any intermediate amount, wherein the total amount of solubilized nimodipine is about 60.00 mg.

In another embodiment, the total pharmaceutical composition volume is less than about 20 mL. For example, less than about 15 mL, less than about 10 mL, less than about 9 mL, less than about 8 mL, less than about 7 mL, less than about 6 mL, less than about 5 mL, or less than about 4 mL. In other embodiments, the present invention provides a pharmaceutical composition of nimodipine, wherein the total composition volume is from about 3 mL to about 20 mL, from about 5 mL to about 15 mL, from about 5 mL to about 12 mL, from about 5 mL to about 10 mL, from about 5 mL to about 8 mL, from about 5 mL to about 6 mL, or from about 7 mL to about 12 mL. For example, about 4 mL, about 5 mL, about 6 mL, about 7 mL, about 8 mL, about 9 mL, about 10 mL, about 11 mL, or about 12 mL.

In another embodiment, the present invention provides a composition of nimodipine, wherein the concentration of nimodipine is at least 5.00 mg/mL. For example, at least 6.00 mg/mL nimodipine, at least 7.00 mg/mL nimodipine, at least 8.00 mg/mL nimodipine, at least 9.00 mg/mL nimodipine, at least 10.00 mg/mL nimodipine, at least 11.00 mg/mL nimodipine, or at least 12.00 mg/mL nimodipine. In other embodiments, the present invention provides a composition of nimodipine, wherein the concentration of nimodipine is from about 5.00 to 10.00 mg/mL, from about 6.00 to about 10.00 mg/mL, from about 7.00 to about 10.00 mg/mL, from about 5.00 to about 20.00 mg/mL, from about 7.00 to about 20.00 mg/mL, from about 8.00 to about 20.00 mg/mL, from about 10.00 to about 20.00 mg/mL, from about 12.00 to about 20.00 mg/mL, or from about 10.00 to about 30.00 mg/mL. For example, about 6.00 mg/mL, about 7.00 mg/mL, about 8.00 mg/mL, about 9.00 mg/mL, about 10.00 mg/mL, about 11.00 mg/mL, about 12.00 mg/mL, about 13.00 mg/mL, about 14.00 mg/mL, or about 15.00 mg/mL.

In another embodiment, the pharmaceutical compositions of the present invention comprise from about 5 percent to about 50 percent alcohol by weight. For example, nimodipine compositions comprise about 5.00, 10.00, 15.00, 20.00, 25.00, 30.00, 35.00, 40.00, 45.00, or about 50.00 percent alcohol, or any intermediate amount, by weight. In another embodiment, a nimodipine composition comprises from about 5 percent to about 50 percent, from about 5 percent to about 40 percent, from about 5 percent to about 30 percent, from about 5 percent to about 20 percent, from about 5 percent to about 15 percent, from about 10 percent to about 40 percent, or from about 10 percent to about 30 percent alcohol by weight. The percent of alcohol is calculated by the total amount of all alcohols (e.g., ethanol and propylene glycol) present in the composition and does not include solvents as described above.

In another embodiment, a composition of nimodipine comprises from about 5 percent to about 20 percent ethanol by weight. For example, about 5.00, 6.00, 7.00, 8.00, 9.00, 10.00, 11.00, 12.00, 13.00, 14.00, 15.00, 16.00, 17.00, 18.00, 19.00, or about 20.00 percent ethanol, or any intermediate amount, by weight. In another embodiment, a composition of nimodipine comprises from about 5 percent to about 20 percent, from about 5 percent to about 10 percent, from about 8 percent to about 12 percent, from about 10 percent to about 20 percent, from about 7 percent to about 15 percent, from about 5 percent to about 15 percent, or from about 5 percent to about 12 percent ethanol by weight.

In another embodiment, the pharmaceutical compositions of the present invention comprise from about 10 percent to about 60 percent solvent by weight. For example, nimodipine compositions comprise about 10.00, 15.00, 20.00, 25.00, 30.00, 35.00, 40.00, 45.00, 50.00, 55.00, or about 60.00 percent solvent, or any intermediate amount, by weight. In another embodiment, a nimodipine composition comprises from about 10 percent to about 60 percent, from about 10 percent to about 50 percent, from about 10 percent to about 40 percent, from about 10 percent to about 30 percent, from about 20 percent to about 60 percent, from about 20 percent to about 50 percent, from about 20 percent to about 40 percent, from about 30 percent to about 60 percent, from about 30 percent to about 50 percent, or from about 40 percent to about 60 percent solvent by weight. The percent of solvent is calculated by the total amount of all solvents (e.g., Cremophor® EL and polyethylene glycol) present in the composition and does not include alcohols as described above.

In another embodiment, the pharmaceutical compositions of the present invention comprise from about 20 percent to about 60 percent water by weight. For example, nimodipine compositions comprise about 20.00, 25.00, 30.00, 35.00, 40.00, 45.00, 50.00, 55.00, or about 60.00 percent water, or any intermediate amount, by weight. In another embodiment, a nimodipine composition comprises from about 20 percent to about 60 percent, from about 20 percent to about 50 percent, from about 20 percent to about 40 percent, from about 20 percent to about 30 percent, from about 30 percent to about 60 percent, from about 30 percent to about 50 percent, from about 30 percent to about 40 percent, from about 40 percent to about 60 percent, from about 40 percent to about 50 percent, from about 50 percent to about 60 percent, from about 20 percent to about 25 percent, from about 25 percent to about 30 percent, from about 30 percent to about 35 percent, or from about 35 percent to about 40 percent water by weight.

In another embodiment, a pharmaceutical composition of nimodipine comprises nimodipine, an alcohol, and a solvent, wherein the concentration of nimodipine is at least about 5 mg/mL. In a specific embodiment, the alcohol is ethanol. In another specific embodiment, the solvent is a non-ionic surfactant, such as a Cremophor® brand non-ionic surfactant. In another specific embodiment, the solvent is Cremophor® EL.

In another embodiment, a pharmaceutical composition of nimodipine comprises nimodipine, an alcohol, and a solvent, wherein the concentration of nimodipine is from about 5 mg/mL to about 20 mg/mL.

In another embodiment, a composition of the present invention further comprises a buffer system. For example, a citrate buffer may be used to maintain and/or control pH conditions.

Pharmaceutical compositions of the present invention possess a viscosity which is appropriate for administration via an intraoral or an intranasal (e.g., naso-gastric) tube. More specifically, in order to enable administration of nimodipine compositions via a naso-gastric tube, the viscosity of the composition must be low enough to allow such flow. This is achieved by the improved combinations of components set forth in the compositions described herein and their relative ratios. The present invention includes such compositions with appropriate viscosity characteristics for the administration methods described herein.

In another embodiment, the present invention provides a pharmaceutical composition of nimodipine, wherein said composition is prepared in a readily-dispensable container. A readily-dispensable container can perform two functions, first, it can act as a storage package for the composition between manufacture and administration, and second, it also can be used to administer the composition to a patient in need thereof. Such administration can be accomplished, for example, via intravenous, intraoral, or intranasal administration. For example, some automated medication dispensing systems (e.g., Pyxis MedStation®) used for controlling medication administration, employ readily-dispensable containers. Said readily-dispensable containers can be used for administration of the pharmaceutical composition, for example, via an intraoral or an intranasal (e.g., naso-gastric) tube. In another embodiment, a readily-dispensable container can be equipped with a means for administering its contents directly into an intraoral or an intranasal (e.g., naso-gastric) tube. In another embodiment, a readily-dispensable container can be compatible for use in one or more automated medication dispensing systems.

The nimodipine compositions of the present invention are also suitable for other methods of administration of a liquid to a mammal in need thereof, such as other oral dosage methods (e.g., liquid-filled capsule, elixir, or syrup).

In another embodiment, the present invention provides a 60 mg dose of nimodipine with about a 5-15 mL composition volume packaged in a single-use container. In another embodiment, said 60 mg dose is completely solubilized in a liquid composition. In another embodiment, said single-use container comprises less than or equal to 15.00 mL of a nimodipine composition, such as, for example, less than or equal to 12.00 mL, less than or equal to 10.00 mL, less than or equal to 9.00 mL, less than or equal to 8.00 mL, less than or equal to 7.00 mL, less than or equal to 6.00 mL, or less than or equal to 5.00 mL. For example, the total composition volume can be about 3.00 mL, 4.00 mL, 5.00 mL, 6.00 mL, 7.00 mL, 8.00 mL, 9.00 mL, 10.00 mL, 11.00 mL, 12.00 mL, 13.00 mL, 14.00 mL, 15.00 mL, or any intermediate amount, wherein the total amount of solubilized nimodipine is about 60.00 mg.

In another embodiment, the present invention provides a medicament comprising nimodipine, an alcohol, and a solvent.

In another embodiment, a method of treating a mammal suffering from one or more conditions such as, but not limited to, aneurysms, subarachnoid hemorrhage, vasospastic angina, Prenzmetal's angina, stable angina, acute myocardial infarction, myocardial arrest, arrhythmia, systemic hypertension, pulmonary hypertension, congestive heart failure, and hypertrophic cardiomyopathy is provided, comprising administering to said mammal a composition of nimodipine of the present invention. In another embodiment, said mammal is a human.

Single dosage forms of the invention can comprise a solution of nimodipine in an amount of from about 10.0 mg to about 120.0 mg, from about 20.0 mg to about 90.0 mg, or from about 30.0 mg to about 60.0 mg. In another embodiment of the invention, a pharmaceutical composition comprising nimodipine is administered via an intraoral or an intranasal tube as needed in an amount of from about 10.0 mg to about 120.0 mg, from about 10.0 mg to about 90.0 mg, from about 20.0 mg to about 60.0 mg, or from about 30.0 mg to about 60.0 mg. For example, about 30.0, 35.0, 40.0, 45.0, 50.0, 55.0, or 60.0 mg, or any intermediate amount thereof. The dosage amounts can be administered in single or divided liquid doses. Administration can include multiple dosages over several regular intervals of time, such as one dosage of about 60 mg nimodipine (about 5-15 mL composition volume) every 4 hours for up to 21 days or longer.

Nimodipine compositions of the present invention can further comprise other ingredients, that are not normally considered excipients and which may also be biologically active. For example, a nimodipine composition of the invention may comprise one or more additional active pharmaceutical ingredients (APIs). Alternatively, nimodipine compositions of the invention may be co-administered with one or more such additional APIs.

Liquid nimodipine compositions of the present invention can further comprise one or more sweeteners known in the art, such as, but not limited to, saccharin, sucrose, or sucralose.

Although not necessary to practice the present invention, the present nimodipine compositions can also comprise an optional antimicrobial agent. For example, a composition of the invention can comprise disodium edetate, metabisulfate, or a preservative such as benzyl alcohol, or an antioxidant such as cysteine or a salt thereof to retard the growth of microorganisms.

Another embodiment includes a sterile pharmaceutical composition for intranasal or intraoral administration which comprises an aqueous solution of nimodipine, and which further comprises a microbiostatic, microbicidal, preservative, or antioxidant. The nimodipine containing compositions can be provided or administered as sterile pharmaceutical compositions. For example, the nimodipine containing compositions are administered substantially free of microorganisms. The preparation of sterile pharmaceutical compositions is well known to those experienced in the art. Sterile nimodipine containing compositions can be prepared using conventional techniques such as, for example, sterilization of final products or aseptic manufacture. In another embodiment, the sterile compositions of the invention are substantially free of microorganisms for a longer period of time after opening than currently available nimodipine compositions.

Aqueous compositions of the invention can be clear, transparent, and sterile, or they can be readily sterilized by conventional and routine methods such as ultrafiltration. Moreover, several compositions of the invention are both chemically and physically stable over a wide range of environmental conditions, including a range of different temperatures and pH conditions (about pH 5-7).

The compositions of the invention do not exhibit substantial nimodipine degradation such as, for example, no more than about 5% or no more than about 3% loss of nimodipine potency at room temperature over a given study period. The chemical stability can affect important characteristics of the nimodipine composition including shelf-life, proper storage conditions, acceptable environments for administration, biological compatibility, and effectiveness of the nimodipine. Chemical stability can be assessed using techniques well known in the art. For example, assays to detect degradation information obtained from stress studies (e.g., products of acid and base hydrolysis, thermal degradation, photolysis, and oxidation) for both active ingredients and excipients are numerous. One example of a technique that can be used to assess chemical stability is reverse phase high performance liquid chromatography (HPLC).

Alternatively, nimodipine degradation can be assessed by measuring nimodipine degradate concentrations. In some embodiments, the compositions do not exhibit substantial increases in nimodipine degradates such as, for example, no more than about 0.05%, no more than about 0.1%, or no more than about 0.2% increase in nimodipine degradate concentration over a given study period. In another embodiment, any single degradate does not exceed the International Conference on Harmonization (ICH) guidelines, unless specific qualification of that degradate has been performed. (See ICH Document Q3B).

In one embodiment, the liquid compositions do not experience substantial nimodipine degradation for a period of at least about 6 months when stored refrigerated. In another embodiment, the compositions do not experience substantial nimodipine degradation for a period of at least about one year when stored refrigerated. In another embodiment, the compositions do not experience substantial nimodipine degradation for at least about 6 months, for at least about one year, or for at least about two years when stored at or below about room temperature.

The compositions of the present invention preferably have a physiologically neutral pH, such as between about 5.0 and about 7.0. The pH of the nimodipine containing compositions can be adjusted as necessary by, for example, the addition of a base or a salt thereof, for example, an alkali such as sodium hydroxide, potassium hydroxide, or the like. Alternatively, an acid or a salt thereof such as hydrochloric acid, citric acid, or the like can be used to adjust the pH of the compositions.

In some embodiments, the stability of the compositions of this invention are sensitive to pH. In some compositions, nimodipine containing compositions have greater stability at a pH of about 5.0 to 6.0, at about 6.0 to 7.0, at about 5.5 to 6.5, at about 5.0 to 6.5 at about 5.5 to 7.0, or at about 6.5 to 7.0. The pH of the composition can be adjusted with a pharmaceutically acceptable acid or base to obtain a desired pH. In some embodiments, a specific pH can affect the composition stability or microbial growth.

The compositions of the present invention can be provided in forms that possess desired nimodipine concentrations and are ready for direct administration to a patient. Alternatively, compositions can be provided in a concentrated form that requires dilution, for example, with water or an injectable solution, prior to administration.

Compositions of the present invention can be formed by mixing, for example, nimodipine, an alcohol, a solvent, and water. Several methods of mixing the composition components are contemplated and some of these are described below. Alcohol and solvent can be mixed into the compositions as neat components or in water. Nimodipine can be mixed into at least one or more neat components or into at least one or more components in water. The nimodipine may be mixed with at least one or more components in water and then combined with either (1) at least one or more neat components or (2) with at least one or more components mixed in water. In another embodiment, the components are mixed together, water is added with mixing, then nimodipine is added with mixing, and finally, additional water is optionally added to increase the mixture volume. In another embodiment, components in water are mixed together, nimodipine is added with mixing, and finally, additional water is optionally added to increase the mixture volume. In some embodiments, nimodipine is added last.

The water used in the compositions of the present invention is preferably suitable for mammalian, including human, injection. The water should meet appropriate government and/or health care industry standards.

Mixing may be performed by any of the various methods known in the art.

The compositions can be provided, prepared, stored, or transported in any container suitable for maintaining sterility. The container can incorporate means for dispensing an aqueous composition such as, for example, a pierceable or removable seal. The compositions can be dispensed, for example, by loading into an automated medication dispensing system, by extraction with a syringe, or by pouring the composition directly into a device (e.g., a syringe or machine) for administration to a patient. Other means for providing, preparing, storing, transporting, and dispensing sterile pharmaceutical compositions are known to those skilled in the art.

It is noted that exemplary amounts or ranges of amounts for nimodipine, alcohol, solvent, and various other ingredients in the compositions of this invention are provided throughout the description of this invention and its various embodiments. However, it will be appreciated by those skilled in the art that the precise amount of an ingredient used is not critical for practicing the invention. Rather, the amount specified for any ingredient in the description of this invention is merely approximate. Compositions containing about the same amount of a particular ingredient can also be used, even when the words “about” and/or “approximate” are not used here to describe an amount of that ingredient.

### EXEMPLIFICATION

### Example 1

Nimodipine Composition

A nimodipine composition was prepared according to the following:

1. 1 liter of 10 mM citrate buffer was prepared at pH 7.0. 2.0 grams of anhydrous citric acid was added to 1 liter of distilled water, stirred, and dissolved completely. The pH was adjusted to 7.0 with 5 N sodium hydroxide solution.

2. 1 liter of 40 percent (v/v) ethanol in 10 mM citrate buffer was prepared by mixing ethanol (400 mL) with 10 mM citrate buffer (600 mL) from step 1.

3. To prepare a 1 liter batch of the composition, Cremophor® EL (30 percent w/w, 300 grams) was added to the 40 percent ethanol/citrate buffer (69 percent w/w, 690 grams) solution from step 2. The container was then covered and mixed well by stirring for 10 to 20 minutes.

4. 10 mg of Sucralose was added to the solution from step 3. The container was then covered and stirred until complete dissolution.

5. 10 grams of nimodipine powder was added to the solution from step 4. The container was mixed with high speed stirring to complete dissolution. The container was sealed and covered with aluminum foil to protect from light.

6. The solution was visually inspected for any undissolved materials.

The nimodipine composition was stored in the sealed container with protection from light. The composition is stable at room temperature (22 degrees C.). Table 1 includes a description of the composition.

TABLE 1
Nimodipine composition

| Composition Component | Concentration | |
|---|---|---|
| Nimodipine | 10 | mg/mL |
| Cremophor ® EL | 30% | w/w |
| Ethanol | 28% | v/v |
| Sucralose | 0.1% | w/w |
| Citrate buffer | 10 | mM |

| | |
|---|---|
| Distilled water | the rest |
| | Final pH = 7.0 |

### Example 2

Nimodipine Composition Stability

The chemical stability of nimodipine was measured at 40 and 60 degrees C. via HPLC in a composition described in Table 2.

TABLE 2
Nimodipine chemical stability composition

| Composition Component | Concentration | |
|---|---|---|
| Nimodipine | 3.0 | mg/mL |
| Vitamin E TPGS | 10% | w/w |
| Cremophor ® EL | 10% | w/w |
| Citric acid | 10 | mM |

| | |
|---|---|
| Water | the rest |

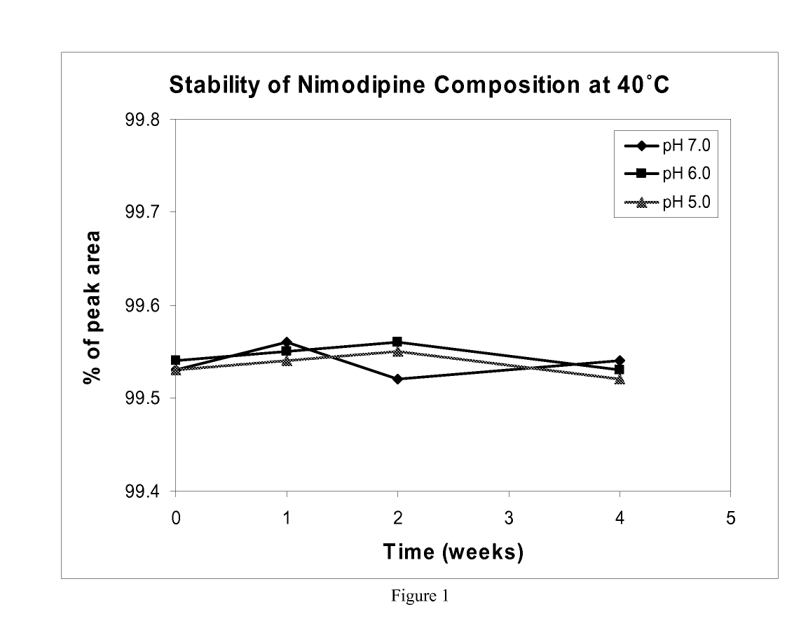

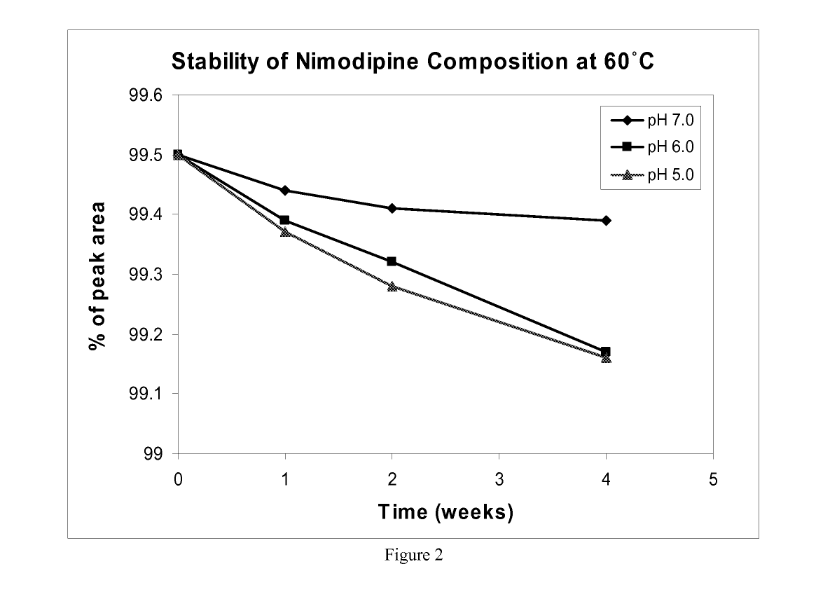

FIGS. 1 and 2 show the chemical stability of nimodipine in the composition described in Table 2 at 40 and 60 degrees C., respectively. These Figures show data acquired with compositions having a pH of 5, 6, and 7, over a period of four weeks. In all cases, nimodipine stability has been shown to be greater than 99 percent. Subsequent data has also been acquired over a period of 8 weeks. The 8 week data also shows greater than 99 percent stability of nimodipine at 60 degrees C. and pH=7.

### Example 3

Solubility Studies of Nimodipine

The effect of several alcohol and solvent concentrations on nimodipine solubility in water was studied. Nimodipine equilibrium solubility was tested at room temperature with the following solutions:
1. Various % (v/v) of ethanol in 10 mM pH 6.8 citrate buffer
2. Various % (w/w) of Cremophor® EL in 10 mM pH 6.8 citrate buffer
3. Various % (v/v) of ethanol with 30% (w/w) Cremophor® EL in 10 mM pH 6.8 citrate buffer
4. Various % (w/w) of propylene glycol (PG) in the solution of 30% Cremophor™ EL and 10% ethanol in citrate buffer at pH 6.8
5. Various % (w/w) of propylene glycol (PG) in the solution of 20% Cremophor® EL and 10% ethanol in citrate buffer at pH 6.8
6. Various % (w/w) of PEG 400 in the solution of 20% Cremophor® EL and 10% ethanol in citrate buffer at pH 6.8
7. Various % (w/w) of PEG400 in the solution of 30% Cremophor® EL and 10% ethanol in citrate buffer at pH 6.8
8. Various % (w/w) of mixture of PG/PEG400 at 1:1 (w/w) in the solution of 30% Cremophor® EL and 10% ethanol in citrate buffer at pH6.8

The above solutions were prepared by weight except solution 1 which the percent ethanol was mixed with citrate buffer by volume.

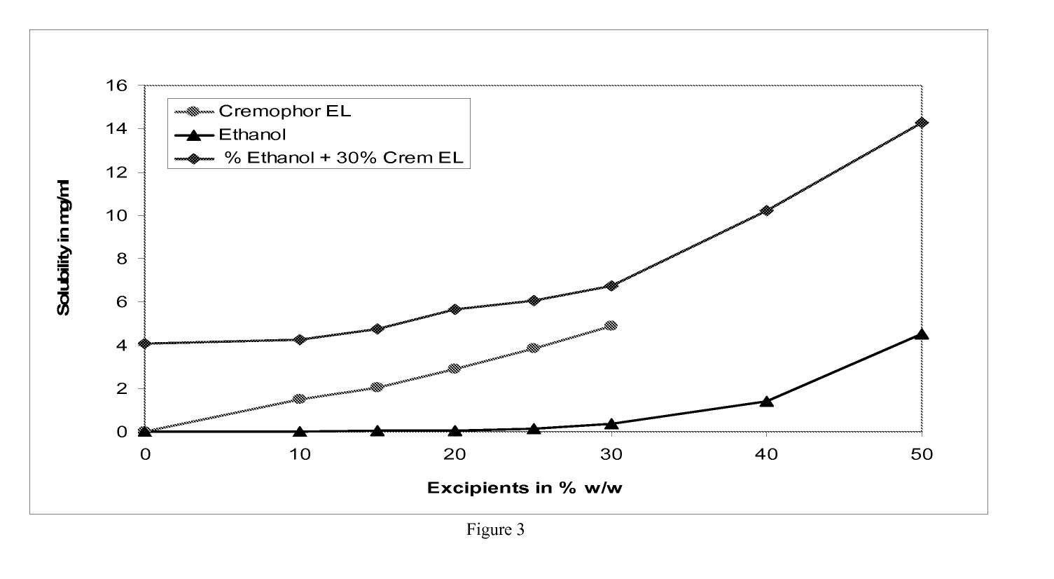

The solubility of nimodipine in above solutions was tested with excess amount of nimodipine and constant stirring for 24 hours at room temperature, then centrifuged at 10 K rpm for 5 minutes. 15˜20 μl of supernatant of each sample was transferred and diluted with water in vials for HPLC analysis. Concentration of each sample was determined by HPLC based on a standard curve and the dilution factor of each samples solution. FIGS. 3-7 show the nimodipine solubility in various alcohol/solvent/water mixtures.

In order to illustrate the advantages of the present invention over liquid nimodipine compositions with high ethanol concentrations (about 20 percent or more by weight), FIG. 3 provides solubility data with compositions comprising up to 50 percent ethanol by weight. FIG. 3 shows several aqueous mixtures of Cremophor® EL, ethanol, and ethanol/Cremophor® EL with varying amounts of excipient. For example, in FIG. 3 the shaded circle data represent aqueous mixtures of Cremophor® EL with an increasing weight percent of the excipient (Cremophor® EL) from 0 to 30 percent, the shaded triangle data represent aqueous mixtures of ethanol with an increasing weight percent of the excipient (ethanol) from 0 to 50 percent, and the shaded diamond data represent aqueous mixtures of ethanol and 30 percent Cremophor® EL by weight with an increasing weight percent of the excipient (ethanol) from 0 to 50 percent.

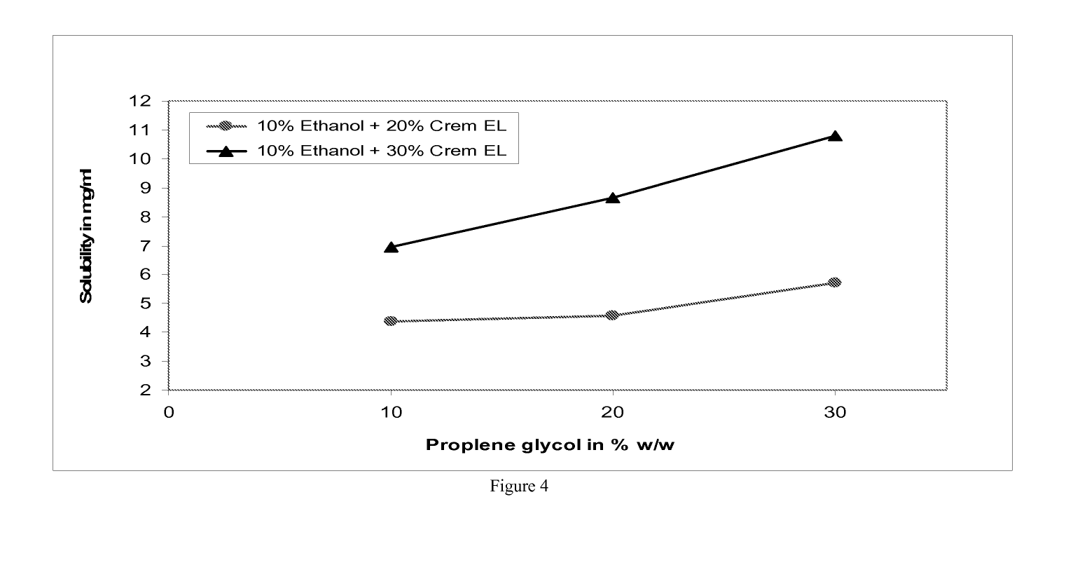

FIG. 4 shows several aqueous mixtures, according to this invention, of ethanol, Cremophor® EL, and propylene glycol with varying amounts of propylene glycol. For example, in FIG. 4 the shaded circle data represent aqueous mixtures of 10% ethanol, 20% Cremophor® EL, and propylene glycol with an increasing weight percent of propylene glycol from 10 to 30 percent, while the shaded triangle data represent aqueous mixtures of 10% ethanol, 30% Cremophor® EL, and propylene glycol with an increasing weight percent of propylene glycol from 10 to 30 percent.

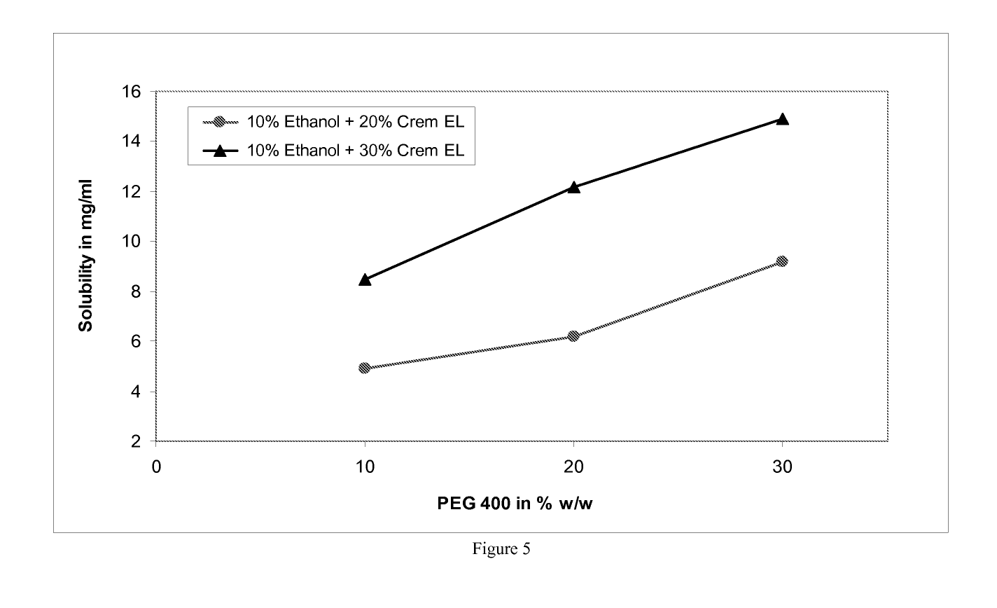

FIG. 5 shows several aqueous mixtures, according to this invention, of ethanol, Cremophor® EL, and PEG 400 with varying amounts of PEG 400. For example, in FIG. 5 the shaded circle data represent aqueous mixtures of 10% ethanol, 20% Cremophor® EL, and PEG 400 with an increasing weight percent of PEG 400 from 10 to 30 percent, while the shaded triangle data represent aqueous mixtures of 10% ethanol, 30% Cremophor® EL, and PEG 400 with an increasing weight percent of PEG 400 from 10 to 30 percent.

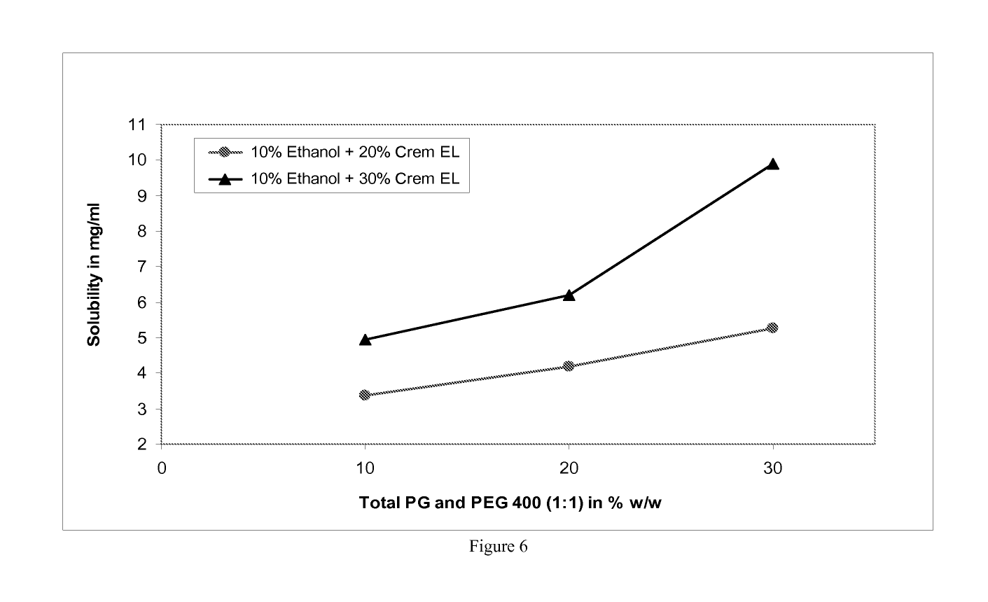

FIG. 6 shows several aqueous mixtures, according to this invention, of ethanol, Cremophor® EL, and 1:1 propylene glycol:PEG 400 with varying amounts of 1:1 propylene glycol:PEG 400. For example, in FIG. 6 the shaded circle data represent aqueous mixtures of 10% ethanol, 20% Cremophor® EL, and 1:1 propylene glycol:PEG 400 with an increasing weight percent of 1:1 propylene glycol:PEG 400 from 10 to 30 percent, while the shaded triangle data represent aqueous mixtures of 10% ethanol, 30% Cremophor® EL, and 1:1 propylene glycol:PEG 400 with an increasing weight percent of 1:1 propylene glycol:PEG 400 from 10 to 30 percent.

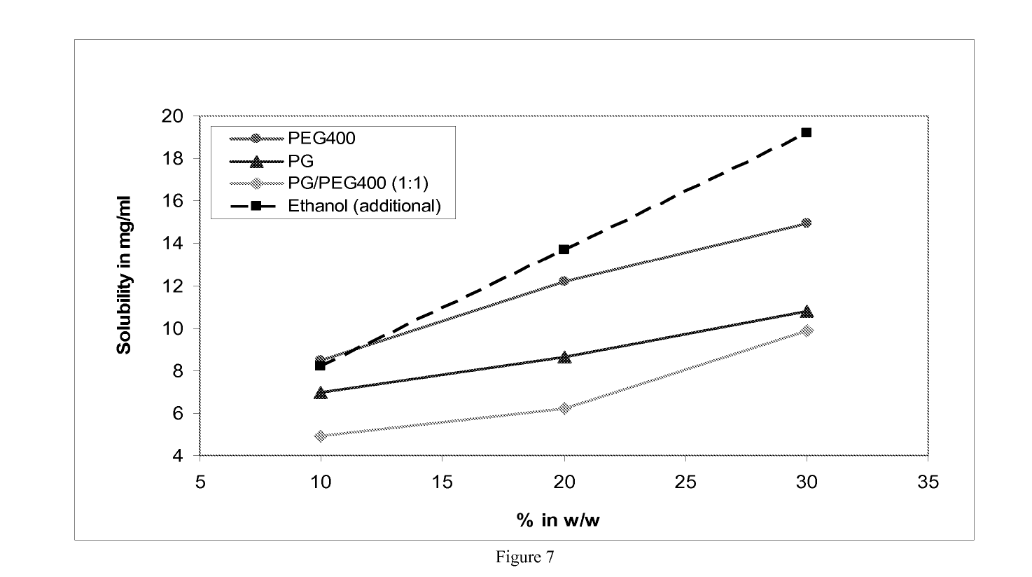

FIG. 7 shows several aqueous mixtures, according to this invention, of PEG 400, propylene glycol, 1:1 propylene glycol:PEG 400, and ethanol in varying amounts with 10% ethanol and 30% Cremophor® EL. For example, in FIG. 7 the shaded circle data represent aqueous mixtures of 10% ethanol, 30% Cremophor® EL, and PEG 400 with an increasing weight percent of PEG 400 from 10 to 30 percent, the shaded triangle data represent aqueous mixtures of 10% ethanol, 30% Cremophor® EL, and propylene glycol with an increasing weight percent of propylene glycol from 10 to 30 percent, the shaded diamond data represent aqueous mixtures of 10% ethanol, 30% Cremophor® EL, and 1:1 propylene glycol:PEG 400 with an increasing weight percent of 1:1 propylene glycol:PEG 400 from 10 to 30 percent, and the shaded square data represent aqueous mixtures of 10% ethanol, 30% Cremophor® EL, and additional ethanol with an increasing weight percent of ethanol from 10 to 30 percent (20 to 40 percent total ethanol). Based on the data shown in FIG. 7, the composition comprising 10% ethanol, 30% Cremophor® EL, and 30% PEG 400 exhibits a comparable nimodipine solubility to the composition comprising 30% ethanol and 30% Cremophor® EL. This formulation comprises only a third of the ethanol concentration while maintaining a nimodipine solubility of about 14 mg/mL.

HPLC Method Description

| | |
|---|---|
| HPLC system: | Binary pump module (Waters Alliance 2690) |
| | Dual wavelength detector (Waters Alliance 2487) |
| | Empower-control and integration software |
| Column: | Bischoff, Symmetry ® C18 H, 5 μm 3.0 × 150 mm |
| Detection: | Channel 1: 237 nm |
| | Channel 2: 355 nm |
| Injection: | 10 μL |
| Column oven: | 30° C. |
| Loop wash: | Acetonitrile/Water 1/1 (V/V) |
| Autosampler temp.: | 5° C. |
| Mobile phase A: | Water with 0.1% TFA |
| Mobile phase B: | Acetonitrile with 0.1% TFA |
| Flow rate: | 1.0 mL/min |

| Gradient program: | Time (min) | Flow | % A | % B |
|---|---|---|---|---|
| | 0.00 | 1.00 | 70 | 30 |
| | 7.00 | 1.00 | 10 | 90 |
| | 7.50 | 1.00 | 70 | 30 |
| | 15.00 | 1.00 | 70 | 30 |
| | 16.00 | 0.00 | 100 | 0 |
| | 20 | 0.00 | 100 | 0 |

What is claimed is:

1. A liquid pharmaceutical composition comprising:
(a) nimodipine;
(b) an alcohol; and
(c) a solvent;
with the proviso that if said alcohol comprises ethanol, the ethanol concentration of said pharmaceutical composition is less than about 15 percent by weight.

2. The liquid pharmaceutical composition of claim 1, wherein said alcohol comprises ethanol.

3. The liquid pharmaceutical composition of claim 1, wherein said solvent comprises a non-ionic surfactant.

4. The liquid pharmaceutical composition of claim 1, further comprising water.

5. The liquid pharmaceutical composition of claim 1, wherein the concentration of nimodipine is at least about 8 mg/mL.

6. The liquid pharmaceutical composition of claim 1, wherein the concentration of nimodipine is at least about 10 mg/mL.

7. The liquid pharmaceutical composition of claim 1, wherein the concentration of nimodipine is from about 10 mg/mL to about 20 mg/mL.

8. The liquid pharmaceutical composition of claim 1, wherein the amount of nimodipine is about 60 mg.

9. The liquid pharmaceutical composition of claim 8, wherein said composition volume is from about 5 mL to about 12 mL.

10. The liquid pharmaceutical composition of claim 8, wherein said composition volume is from about 5 mL to about 10 mL.

11. The liquid pharmaceutical composition of claim 8, wherein said composition volume is from about 5 mL to about 7 mL.

12. The liquid pharmaceutical composition of claim 8, wherein said composition volume is about 5 mL.

13. The liquid pharmaceutical composition of claim 1, wherein said composition is prepared in a readily-dispensable container suitable for an automated medication dispensing system.

14. The liquid pharmaceutical composition of claim 13, wherein said readily-dispensable container is compatible with a naso-gastric tube for intranasal administration.

15. A method of treating a mammal suffering from one or more conditions such as, but not limited to, aneurysms, subarachnoid hemorrhage, vasospastic angina, Prenzmetal's angina, stable angina, acute myocardial infarction, myocardial arrest, arrhythmia, systemic hypertension, pulmonary hypertension, congestive heart failure, and hypertrophic cardiomyopathy, comprising administering to said mammal the liquid pharmaceutical composition of claim 1.
